(19) United States
(12) Patent Application Publication
(10) Pub. No.: US 2026/0174357 A1
(43) Pub. Date: Jun. 25, 2026

(54) PSEUDO ABNORMAL DATA GENERATING DEVICE, FACILITY MONITORING SYSTEM, PSEUDO ABNORMAL DATA GENERATING METHOD, AND NON-TRANSITORY COMPUTER-READABLE MEDIUM

(71) Applicant: Mitsubishi Electric Corporation, Chiyoda-ku, Tokyo (JP)

(72) Inventor: Kazuki FUJII, Tokyo (JP)

(73) Assignee: Mitsubishi Electric Corporation, Chiyoda-ku, Tokyo (JP)

(21) Appl. No.: 18/726,883

(22) PCT Filed: Jan. 20, 2023
(86) PCT No.: PCT/JP2023/001616

§ 371 Date: Jul. 5, 2024

(51) Int. Cl. A61B 5/11

(52) U.S. Cl. CPC A61B 5/1114

(57) ABSTRACT

There is provided a pseudo abnormal data generating device including: a group generating unit configured to generate a group including at least a part of a plurality of pieces of abnormal data on the basis of at least feature quantities of the plurality of pieces of abnormal data from the plurality of pieces of abnormal data that is time series data; a band generating unit configured to generate a band representing a range of values including abnormal data included in the group generated by the group generating unit at each time; and a band modulating unit configured to generate pseudo abnormal data on the basis of the band generated by the band generating unit. In accordance with this, pseudo abnormal data can be generated without using knowhow for performing a simulation and an experiment.

### TECHNICAL FIELD

The present invention relates to a pseudo abnormal data generating device, a facility monitoring system, a pseudo abnormal data generating method, and a non-transitory computer-readable medium.

### BACKGROUND ART

A learning model for detecting an abnormality of a facility device is generated using machine learning. However, since the amount of data at the time of occurrence of an abnormality is small, a technology for generating pseudo abnormal data used for generating a learning model detecting an abnormality has been disclosed (see Patent Document 1). In this method of generating pseudo abnormal data, a simulation based on a physical model of facilities, an experiment in which a constituent component is intentionally degraded or broken, and the like are used.

### CITATION LIST

### Patent Document

[Patent Document 1]

Japanese Unexamined Patent Application, First Publication No. 2019-133212

### SUMMARY OF INVENTION

### Technical Problem

However, in the method of generating pseudo abnormal data described above, there is a problem in that know-how for performing a simulation or an experiment is necessary.

The present invention is in view of such a situation and provides a pseudo abnormal data generating device, a facility monitoring system, a pseudo abnormal data generating method, and a non-transitory computer-readable medium generating pseudo abnormal data without using know-how for performing a simulation and an experiment.

### Solution to Problem

According to one aspect of the present invention, there is provided a pseudo abnormal data generating device including: a group generating unit configured to generate a group including at least a part of a plurality of pieces of abnormal data on the basis of at least feature quantities of the plurality of pieces of abnormal data from the plurality of pieces of abnormal data that is time series data; a band generating unit configured to generate a band representing a range of values including abnormal data included in the group generated by the group generating unit at each time; and a band modulating unit configured to generate pseudo abnormal data on the basis of the band generated by the band generating unit.

According to another aspect of the present invention, in the pseudo abnormal data generating device described above, the band generating unit adjusts positions of pieces of abnormal data included in the group on the basis of feature quantities of the pieces of abnormal data included in the group and generates the band including the pieces of abnormal data of which positions have been adjusted.

According to another aspect of the present invention, in the pseudo abnormal data generating device described above, the band modulating unit generates the pseudo abnormal data by applying a change to the maximum value or the minimum value of the range of the values represented by the band generated by the band generating unit with the maximum value or the minimum value set as a reference.

According to another aspect of the present invention, in the pseudo abnormal data generating device described above, the band modulating unit performs the change applied to the maximum value or the minimum value at a time of a part of the band.

According to another aspect of the present invention, there is provided a facility monitoring system including: a group generating unit configured to generate a group including at least a part of a plurality of pieces of abnormal data on the basis of at least feature quantities of the plurality of pieces of abnormal data from the plurality of pieces of abnormal data that is time series data; a band generating unit configured to generate a band representing a range of values including abnormal data included in the group generated by the group generating unit at each time; and a band modulating unit configured to generate pseudo abnormal data on the basis of the band generated by the band generating unit.

According to another aspect of the present invention, in the facility monitoring system described above, a learning model generating unit configured to generate a learning model used for detecting an abnormality or a sign of an abnormality from operating data using the pseudo abnormal data and an abnormal sign detecting unit configured to detect an abnormality or a sign of an abnormality from the operating data using the learning model generated by the learning model generating unit are further included.

According to another aspect of the present invention, in the facility monitoring system described above, a sensor unit configured to measure operating data including the abnormal data is further included.

According to another aspect of the present invention, in the facility monitoring system described above, a sensor unit configured to measure operating data including the abnormal data and a data collecting unit configured to collect operating data measured by the sensor unit are further included.

According to another aspect of the present invention, there is provided a pseudo abnormal data generating method including: a first step of generating a group including at least a part of a plurality of pieces of abnormal data on the basis of at least feature quantities of the plurality of pieces of abnormal data from the plurality of pieces of abnormal data that is time series data; a second step of generating a band representing a range of values including abnormal data included in the group generated in the first step at each time; and a third step of generating pseudo abnormal data on the basis of the band generated in the second step.

According to another aspect of the present invention, there is provided a program causing a processor to function as: a group generating unit configured to generate a group including at least a part of a plurality of pieces of abnormal data on the basis of at least feature quantities of the plurality of pieces of abnormal data from the plurality of pieces of abnormal data that is time series data; a band generating unit configured to generate a band representing a range of values including abnormal data included in the group generated by the group generating unit at each time; and a band modulating unit configured to generate pseudo abnormal data on the basis of the band generated by the band generating unit.

### Advantageous Effects of Invention

A pseudo abnormal data generating device according to the present invention can generate pseudo abnormal data without using know-how used for performing a simulation and an experiment.

### BRIEF DESCRIPTION OF DRAWINGS

FIG. 1 is a schematic block diagram illustrating a configuration of a transmission/distribution network monitoring system 10 according to a first embodiment of the present invention.

FIG. 2 is a schematic block diagram illustrating a configuration of a pseudo abnormal data generating device 300 according to the embodiment.

FIG. 3 is a flowchart showing an operation example of an abnormal waveform extracting unit 301 according to the embodiment.

FIG. 4 is a schematic view showing calculation of a score using the abnormal waveform extracting unit 301 according to the embodiment.

FIG. 5 is a flowchart showing an operation example of a clustering unit 303 according to the embodiment.

FIG. 6 is a flowchart showing an operation example of a band generating unit 304 according to the embodiment.

FIG. 7 is a diagram showing an example of position adjustment using the band generating unit 304 according to the embodiment.

FIG. 8 is a diagram showing an example of a band generated by the band generating unit 304 according to the embodiment.

FIG. 9 is a diagram showing an example of band modulation using the band modulating unit 305 according to the embodiment.

FIG. 10 is a diagram illustrating an example of an operation screen for the band modulating unit 305 according to the embodiment.

FIG. 11 is a diagram showing an operation example of a waveform synthesizing unit 307 according to the embodiment.

FIG. 12 is a table illustrating an output example of a pseudo abnormal data output unit 306 according to the embodiment.

FIG. 13 is a schematic block diagram illustrating a configuration of a pseudo abnormal data generating device 300 according to a second embodiment of the present invention.

FIG. 14 is an explanatory diagram showing a hardware configuration of each device according to each embodiment.

### DESCRIPTION OF EMBODIMENTS

### First Embodiment

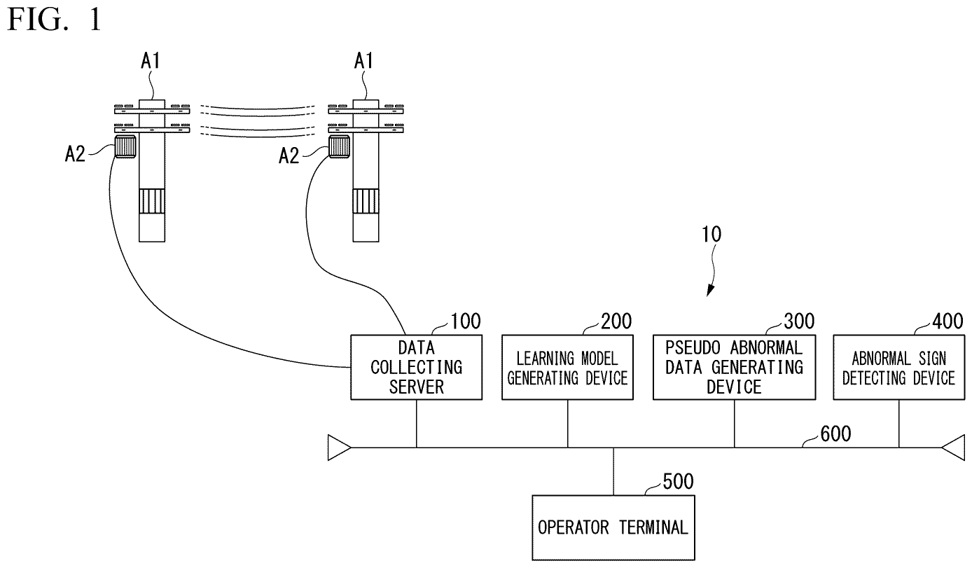

Hereinafter, embodiments of the present invention will be described with reference to the drawings. FIG. 1 is a schematic block diagram illustrating a configuration of a transmission/distribution network monitoring system 10 according to a first embodiment of the present invention. The transmission/distribution network monitoring system 10 (a facility monitoring system) collects operating data measured by a sensor (a sensor unit) installed in a facility such as a relay device A2 or the like disposed on an electricity pole Al and monitors a state of a transmission/distribution network. The operating data measured by a sensor is time-series data and, for example, is a voltage or a current of a distribution line in the relay device A2 at regular time intervals.

The transmission/distribution network monitoring system 10 includes a data collecting server 100 (a data collecting unit), a learning model generating device 200 (a learning model generating unit), a pseudo abnormal data generating device 300, an abnormal sign detecting device 400 (an abnormal sign detecting unit), and an operator terminal 500. The data collecting server 100, the learning model generating device 200, the pseudo abnormal data generating device 300, the abnormal sign detecting device 400, and the operator terminal 500 are connected so as to be able to communicate with each other using a network 600 such as a local area network (LAN) or the like. The data collecting server 100, the learning model generating device 200, the pseudo abnormal data generating device 300, the abnormal sign detecting device 400, and the operator terminal 500 may be realized by a computer reading and executing a program. Each of the data collecting server 100, the learning model generating device 200, the pseudo abnormal data generating device 300, the abnormal sign detecting device 400, and the operator terminal 500 may be realized by one computer or a plurality of computers. In addition, a plurality of devices or some of the plurality of devices may be realized by one computer.

The data collecting server 100 collects and stores operating data measured by a sensor. The learning model generating device 200 generates a learning model for detecting abnormal data from the operating data. In addition, after the pseudo abnormal data generating device 300 generates pseudo abnormal data, when a learning model is generated, the learning model generating device 200 may be configured to use pseudo abnormal data as well in addition to operating data stored by the data collecting server 100.

By using a learning model generated by the learning model generating device 200, the pseudo abnormal data generating device 300 extracts abnormal data from operating data and generates pseudo abnormal data on the basis of the extracted abnormal data. Details of the pseudo abnormal data generating device 300 will be described below.

The abnormal sign detecting device 400 detects abnormal data from operating data using a learning model generated by the learning model generating device 200. When the abnormal data is detected, the abnormal sign detecting device 400 notifies an operator of presence of an abnormality or a sign of an abnormality in a transmission/distribution network through the operator terminal 500. The abnormal sign detecting device 400 may judge a type of detected abnormal data (details will be described below) and notify an operator of this type or a type of abnormality according to this type through the operator terminal 500.

The operator terminal 500 is a terminal used by an operator of the transmission/distribution network monitoring system 10 and performs setting of the transmission/distribution network monitoring system 10 according to an operator, a notification to an operator using the transmission/distribution network monitoring system 10, and the like.

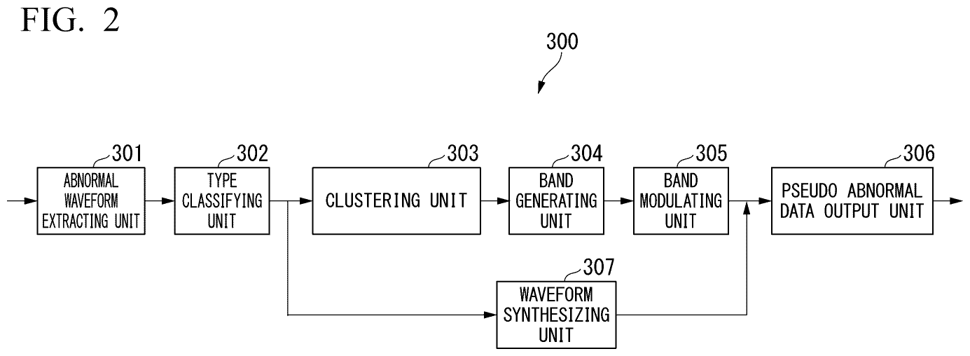

FIG. 2 is a schematic block diagram illustrating a configuration of the pseudo abnormal data generating device 300 according to this embodiment. The pseudo abnormal data generating device 300 includes an abnormal waveform extracting unit 301, a type classifying unit 302, a clustering unit 303, a band generating unit 304, a band modulating unit 305, a pseudo abnormal data output unit 306, and a waveform synthesizing unit 307. In addition, a group generating unit may be configured using the type classifying unit 302 and the clustering unit 303.

The abnormal waveform extracting unit 301 extracts abnormal data (an abnormal waveform) from operating data collected by the data collecting server 100 using a learning model generated by the learning model generating device 200.

The type classifying unit 302 classifies pieces of abnormal data extracted by the abnormal waveform extracting unit 301 into a plurality of types on the basis of feature quantities. Here, as a feature quantity of each piece of abnormal data, at least any one of the average value of the abnormal data, a median value of the abnormal data, crossing times of the abnormal data and the average value or the median value, the difference between the maximum value and the minimum value of the abnormal data, the absolute value of the difference between the value of the abnormal data at a start point and the value of the abnormal data at an end point, the inclination of the abnormal data at a first crossing of the abnormal data and the average value or the median value, or positivity/negativity may be used. In this embodiment, the type classifying unit 302 classifies abnormal data into 6 types including a top peak type, a bottom peak type, a top/bottom peak type, a transient rising type, a transient falling type, and a vibration type using a method described in U.S. Pat. No. 6,827,608.

The clustering unit 303 further classifies abnormal data of each type classified by the type classifying unit 302 into a plurality of clusters (groups). For example, the clustering unit 303 performs classification using a K-means method on the basis of feature quantities such as differences between the maximum value and the minimum value and the like or a distance between abnormal data and abnormal data such as a Euclidean distance or the like.

The band generating unit 304, for each group generated by the clustering unit 303, generates a band that represents a range of values including abnormal data included in the group at each time. In addition, before generating a band, the band generating unit 304 may perform position adjustment in a time direction or a direction of magnitudes of values on the basis of feature quantities of abnormal data. The position adjustment based on feature quantities, for example, is matching of times at which maximum values are acquired, matching of times at which average values are acquired, matching of magnitudes of average values, and the like.

The band modulating unit 305 generates pseudo abnormal data on the basis of each band generated by the band generating unit 304. For example, the band modulating unit 305 sets values acquired by subtracting a random value from a part or the whole of time series data in which maximum values of bands at each time are aligned or values acquired by multiplying a part or the whole of the time series data by a random ratio as pseudo abnormal data. For example, by using a maximum value Max(t), a minimum value Min(t), a random value R(t) of a band at a time t, pseudo abnormal data may be D(t)=Max(t)−R(t) at the time t, here R(t)>0 and R(t)<Max(t)−Min(t).

Alternatively, pseudo abnormal data may be D(t)=Min(t)+R(t)×(Max(t)−Min(t)), here 0≤R(t)≤1.

The pseudo abnormal data output unit 306 outputs pseudo abnormal data generated by the band modulating unit 305 and the waveform synthesizing unit 307. In addition, by displaying a graph of pseudo abnormal data generated by the band modulating unit 305 and the waveform synthesizing unit 307 in the operator terminal 500 or the like, the pseudo abnormal data output unit 306 may output only pseudo abnormal data that has been visually checked by an operator.

The waveform synthesizing unit 307 synthesizes waveform data corresponding to a type into which abnormal data has been classified by the type classifying unit 302 and sets the waveform data as pseudo abnormal data. Which waveform data is synthesized for each type may be set in advance by an operator through the operator terminal 500 or may be set at the time of manufacturing of the pseudo abnormal data generating device 300.

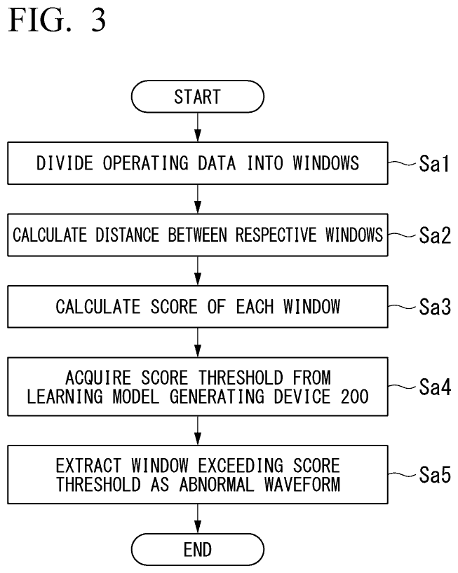

FIG. 3 is a flowchart showing an operation example of the abnormal waveform extracting unit 301 according to this embodiment. First, the abnormal waveform extracting unit 301 divides operating data into windows of a predetermined time length (Step Sa1). The length of this predetermined time may be set in advance through the operator terminal 500 by an operator or may be set at the time of manufacturing the pseudo abnormal data generating device 300.

Next, the abnormal waveform extracting unit 301 calculates a distance between respective windows (Step Sa2). Here, a distance is a value that represents the degree of similarity of time series data configuring windows between the windows, has a smaller value as the degree of similarity becomes higher and for example, and is calculated using a discord (Nakamura, Takaaki et al., “Time Series Data Anomaly Detection Method using Sample Time Series Extraction,” DEIM Forum 2015F8 -2, https://db-event.jpn.org/deim2015/paper/16.pdf).

Next, the abnormal waveform extracting unit 301 calculates a score of each window (Step Sa3). This score has a value that becomes larger as there is no window similar to time series data configuring the window and, for example, sets a smallest value among distances between the window and the other windows as a score.

Next, the abnormal waveform extracting unit 301 acquires a score threshold from the learning model generating device 200 (Step Sa4). This score threshold is a learning model generated by the learning model generating device 200. Similar to Step Sa1 to Step Sa3, the learning model generating device 200 calculates a score of each window of operating data prepared for generating a learning model and determines a score threshold on the basis of a distribution of the calculated scores. For example, the learning model generating device 200 may set a score of a proportion determined in advance from the top among the calculated scores as a score threshold or may set a score that is a deviation value determined in advance as a score threshold.

Next, the abnormal waveform extracting unit 301 extracts a window of which the score calculated in Step Sa3 exceeds the score threshold acquired in Step Sa4 as an abnormal waveform (abnormal data) (Step Sa5).

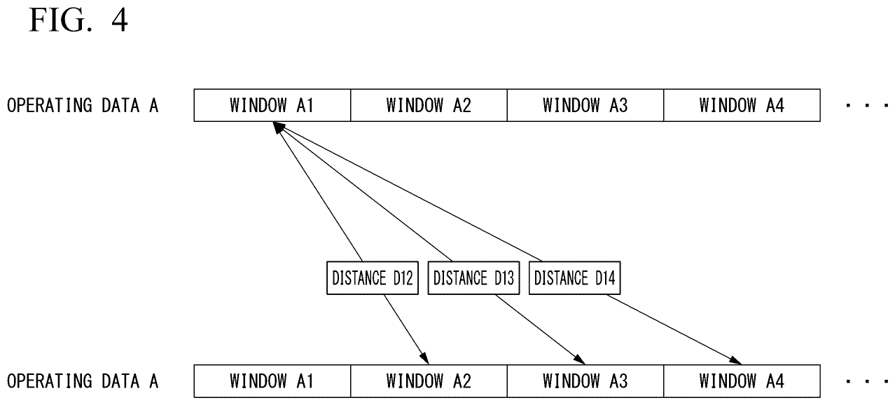

FIG. 4 is a schematic view showing calculation of a score using the abnormal waveform extracting unit 301 according to this embodiment. As illustrated in FIG. 4, the abnormal waveform extracting unit 301 divides operating data A into windows A1, A2, A3, A4 In addition, the abnormal waveform extracting unit 301, for the window A1, calculates a distance D12 to the window A2, a distance D13 to the window A3, a distance D14 to the window A4, . . . and sets the minimum of such distances as a score of the window A1.

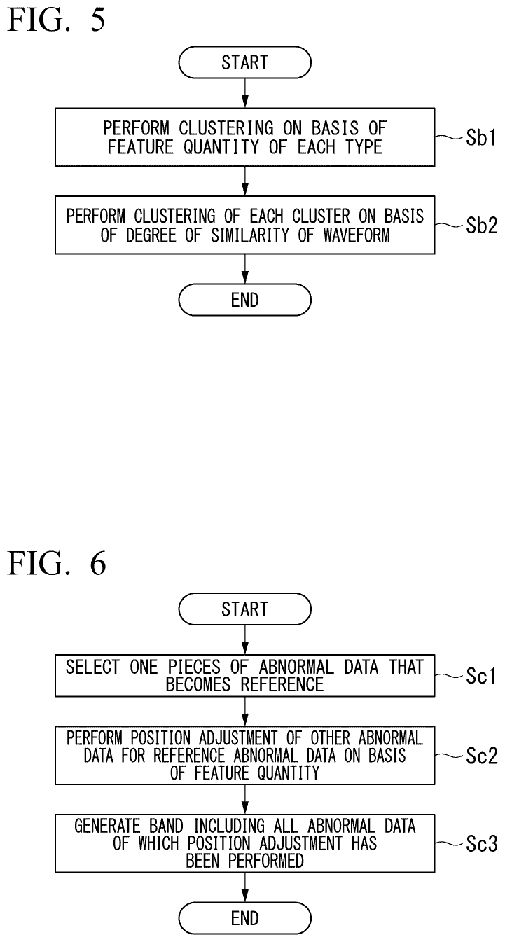

FIG. 5 is a flowchart showing an operation example of the clustering unit 303 according to this embodiment. The clustering unit 303, for abnormal data belonging to each type classified by the type classifying unit 302, performs clustering on the basis of feature quantities thereof (Step Sb1). The feature quantity used at this time may be a feature quantity that corresponds to the type. For example, the top peak type may be clustered on the basis of the maximum value, and the vibration type may be clustered on the basis of a crossing count between the average value and abnormal data. In addition, clustering may be performed on the basis of a plurality of feature quantities.

Next, the clustering unit 303 performs clustering of each cluster that is a result of clustering performed in Step Sb1 on the basis of the degree of similarity between waveforms of abnormal data and generates groups of the abnormal data (Step Sb2). In addition, in a case in which a K means method is used in the clustering of Step Sb1 and Step Sb2, for each of the steps, the value of K may be set by an operator through the operator terminal 500 or may be set at the time of manufacturing the pseudo abnormal data generating device 300.

FIG. 6 is a flowchart showing an operation example of the band generating unit 304 according to this embodiment. The operation example illustrated in FIG. 6 is an example of an operation of the band generating unit 304 for each group generated by the clustering unit 303. First, the band generating unit 304 selects one abnormal waveform that serves as a reference out of abnormal data belonging to the group (Step Sc1). As this selection, for example, a first piece of data out of abnormal data belonging to the group may be selected, abnormal data of which a feature quantity used at the time of generating the group is the closest to the center of the group may be selected, or abnormal data may be designated by an operator through the operator terminal 500.

Next, the band generating unit 304 performs position adjustment of other abnormal data for the reference abnormal data on the basis of the feature quantity (Step Sc2). For example, in the case of a group formed from abnormal data of the top peak type, the band generating unit 304 shifts the other abnormal data in a time direction such that a time at which the other abnormal data becomes the maximum value is the same as a time at which the reference abnormal data becomes the maximum value. In addition, the band generating unit 304 shifts the other abnormal data in a vertical direction (the direction of the value of the abnormal data) such that the average value of the other abnormal data becomes the average value of the reference abnormal data.

Next, the band generating unit 304 generates a band that includes all the abnormal data of which the position adjustment has been performed in Step Sc2 (Step Sc3). For example, the band generating unit 304 extracts minimum values and maximum values of all the abnormal data of which position adjustment has been performed at each time and the reference abnormal data and sets an area surrounded by these as a band.

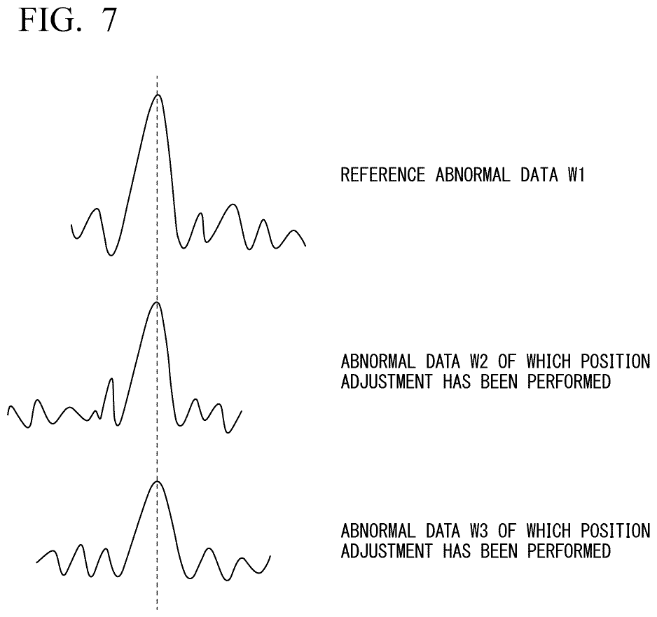

FIG. 7 is a diagram showing an example of position adjustment using the band generating unit 304 according to this embodiment. The example of the position adjustment illustrated in FIG. 7 is an example of position adjustment in a time direction for a group formed from abnormal data of the top peak type. As illustrated in FIG. 7, the band generating unit 304 shifts abnormal data W2 and W3 in the time direction such that a time at which each piece of the abnormal data W2 and W3 becomes the maximum value is the same as a time at which the reference abnormal data W1 becomes the maximum value.

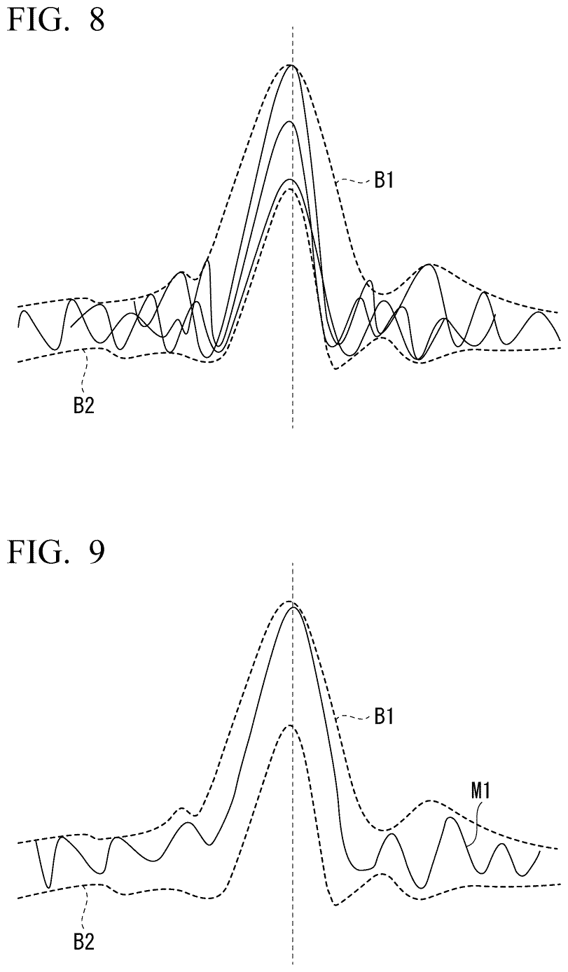

FIG. 8 is a diagram showing an example of a band generated by the band generating unit 304 according to this embodiment. In FIG. 8, a graph B1 is a graph in which the maximum value of abnormal data, of which position adjustment has been performed, at each time is plotted. A graph B2 is a graph in which the minimum value of abnormal data, of which position adjustment has been performed, at each time is plotted. A band generated by the band generating unit 304 becomes an area (range) interposed between the graph B1 and the graph B2.

FIG. 9 is a diagram showing an example of band modulation using the band modulating unit 305 according to this embodiment. In FIG. 9, graphs B1 and B2 are similar to the graph B1 and the graph B2 illustrated in FIG. 8. A graph M1 is pseudo abnormal data. In the example illustrated in FIG. 9, the band modulating unit 305 generates pseudo abnormal data M1 such that it enters a band surrounded by the graph B1 and the graph B2.

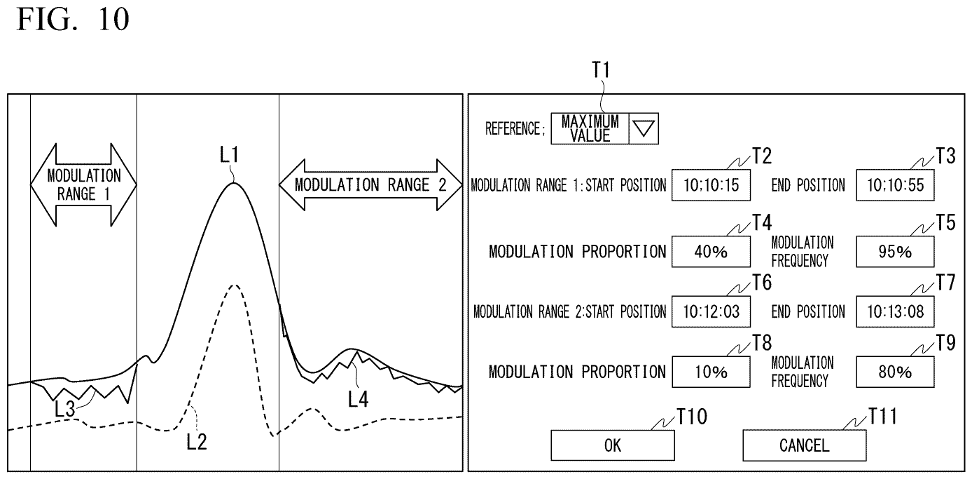

FIG. 10 is a diagram illustrating an example of an operation screen for the band modulating unit 305 according to this embodiment. In order to allow an operator to set parameters at the time of generating pseudo abnormal data, the band modulating unit 305 displays the example of the operation screen illustrated in FIG. 10 in the operator terminal 500. In FIG. 10, input areas T1 to T9 are input areas of parameters for generating pseudo abnormal data. OK button T10 and cancel button T11 are buttons for an operator to designate whether or not generated pseudo abnormal data is to be approved.

The input area T1 is an area for inputting selection of whether a graph, which is used as a reference at the time of generating pseudo abnormal data, is set as the maximum value or the minimum value of the band. The input area T2 is an area to which a start position (a start time) of a range in which the band is modulated (a modulation range 1) is input. The input area T3 is an area to which an end position (an end time) of the range in which the band is modulated (the modulation range 1) is input. The input area T4 is an area to which the maximum value of a proportion changed from a graph serving as a reference through band modulation is input for the modulation range 1. The input area T5 is an area to which a frequency (a proportion) of a time position changed from a graph serving as a reference through band modulation is input for the modulation range 1. The band modulating unit 305 randomly changes the value of the proportion set in the input area T5 among time positions configuring data of the modulation range 1 such that an amount of change is within a proportion set in the input area T4 of the width of the band.

The input area T6 is an area to which a start position (a start time) of a range in which the band is modulated (a modulation range 2) is input. The input area T7 is an area to which an end position (an end time) of a range in which the band is modulated (the modulation range 2) is input. The input area T 8 is an area to which the maximum value of a proportion changed from the graph serving as a reference is changed through band modulation is input for the modulation range 2. The input area T9 is an area to which a frequency (a proportion) of a time position changed from the graph serving as the reference through band modulation is input for the modulation range 2. The band modulating unit 305 randomly changes the value of the proportion set in the input area T9 among time positions configuring data of the modulation range 2 such that the amount of change is within the proportion set in the input area T8 of the width of the band.

A graph L1 is a graph that illustrates a boundary of the maximum value side of the band. A graph L2 is a graph that illustrates a boundary of the minimum value side of the band. In FIG. 10, since the maximum value is selected in the input area T1, the graph L1 is displayed using a solid line, and the graph L2 is displayed using a broken line. A graph L3 illustrated in the modulation range 1 is a graph acquired by changing the proportion set in the input area T4 as the maximum value for the graph L1 in the modulation range 1 at a time position of the frequency set in the input area T5. A graph L4 illustrated in the modulation range 2 is a graph acquired by changing the proportion set in the input area T8 as the maximum value for the graph L1 in the modulation range 2 at a time position of the frequency set in the input area T9.

When the OK button T10 is pressed, the band modulating unit 305 sets a graph formed from the graph L3 for the modulation range 1, the graph L4 for the modulation range 2, and the graph L1 for other times as pseudo abnormal data. When the cancel button T11 is pressed, the band modulating unit 305 discards the pseudo abnormal data of this graph. In FIG. 10, although a case in which two modulation ranges including the modulation range 1 and the modulation range 2 are present has been described as an example, the number of modulation ranges may be one or three or more, or may be set by

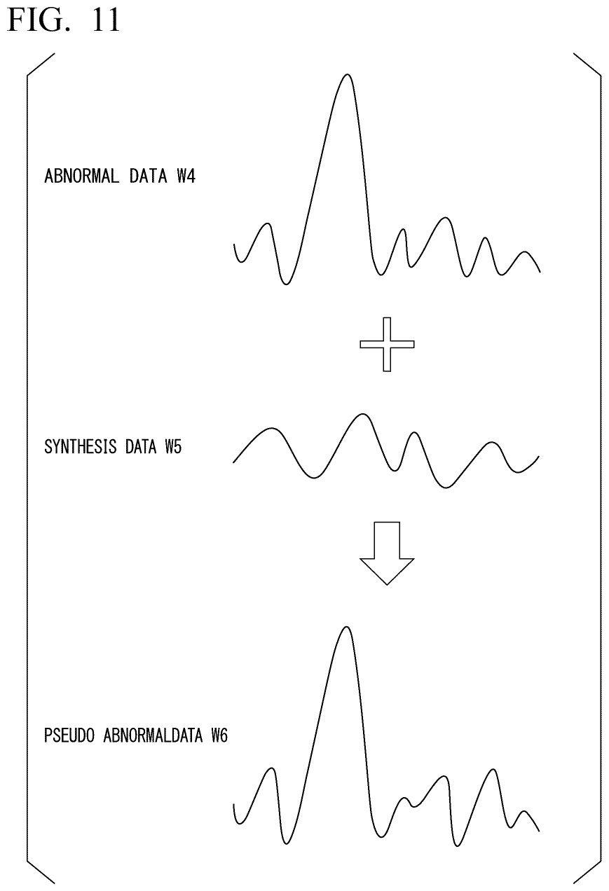

FIG. 11 is a diagram showing an operation example of the waveform synthesizing unit 307 according to this embodiment. As illustrated in FIG. 11, the waveform synthesizing unit 307 adds synthesis data W5 corresponding to the type to abnormal data W4, thereby generating pseudo abnormal data W6. Here, addition represents addition of a value of the abnormal data W4 and a value of the synthesis data W5 at each time. In addition, the synthesis data W5 may be normal data.

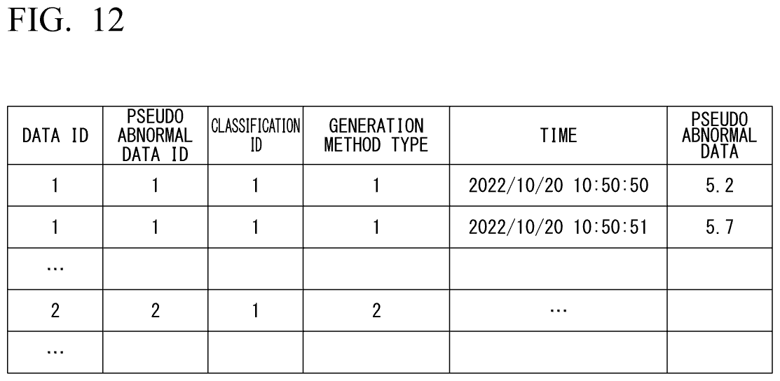

FIG. 12 is a table illustrating an output example of the pseudo abnormal data output unit 306 according to this embodiment. In FIG. 12, a data ID is a number used for identifying operating data that becomes a source at the time of generating pseudo abnormal data or abnormal data. A pseudo abnormal data ID is a number used for identifying pseudo abnormal data. The pseudo abnormal data ID may be assigned such that pseudo abnormal data is uniquely identified in combination with a data ID.

A classification ID is a number that represents a type or a group to which abnormal data that becomes a source at the time of generating pseudo abnormal data belongs. A generation method type is a number that represents whether generation of pseudo abnormal data has been performed through band modulation or waveform synthesis. A time is a time that corresponds to a value of pseudo abnormal data of this row. The pseudo abnormal data is a value of pseudo abnormal data at the time of the same row.

### Second Embodiment

In the first embodiment, synthesis data to be synthesized with abnormal data is determined in accordance with a type classified by the type classifying unit 302 in the waveform synthesizing unit 307. In a second embodiment, synthesis data to be synthesized with abnormal data is determined in accordance with a cluster (a group) according to the clustering unit 303. The configuration of a transmission/distribution network monitoring system 10 according to this embodiment is similar to that illustrated in FIG. 1.

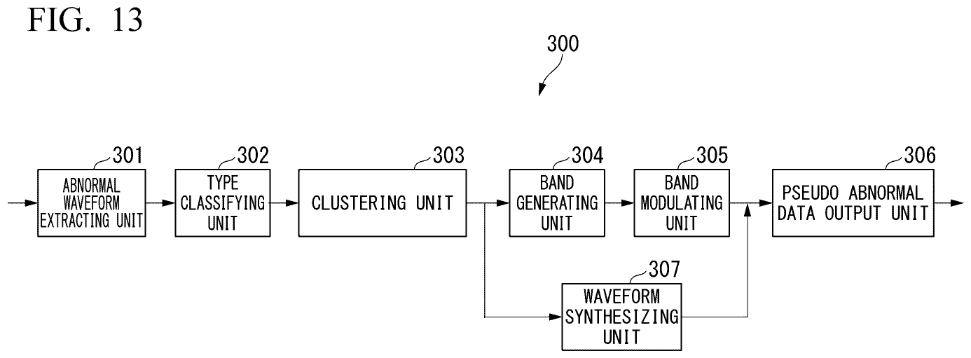

FIG. 13 is a schematic block diagram illustrating a configuration of a pseudo abnormal data generating device 300 according to the second embodiment of the present invention. In FIG. 13, the same reference signs will be assigned to parts corresponding to the parts illustrated in FIG. 2, and description will be omitted. As illustrated in FIG. 13, in the pseudo abnormal data generating device 300 according to this embodiment, a waveform synthesizing unit 307 determines synthesis data on the basis of a cluster generated by a clustering unit 303, which is different from the first embodiment.

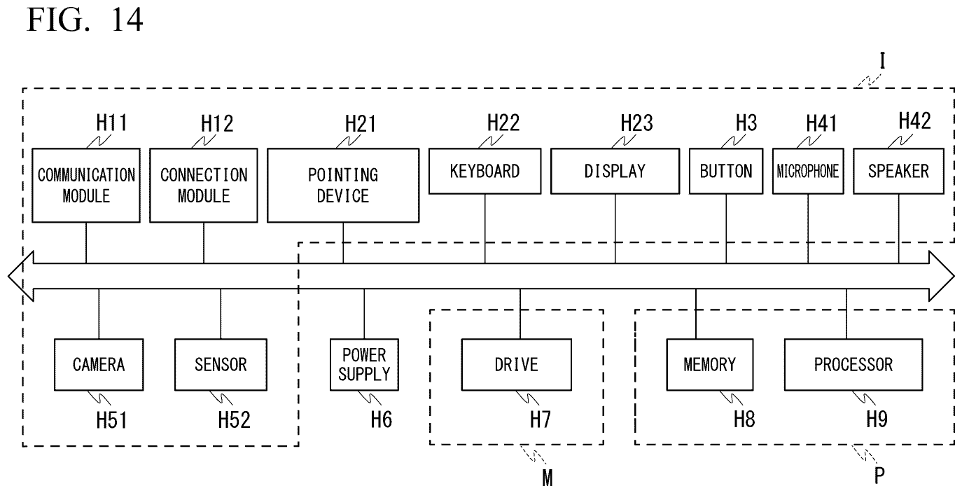

FIG. 14 is an explanatory diagram showing a hardware configuration of each device according to each embodiment described above.

Each device is a data collecting server 100, a learning model generating device 200, a pseudo abnormal data generating device 300, an abnormal sign detecting device 400, and an operator terminal 500. Each device is configured to include an input/output module I, a memory module M, and a control module P. The input/output module I is realized by including some or all of a communication module H11, a connection module H12, a pointing device H21, a keyboard H22, a display H23, a button H3, a microphone H41, a speaker H42, a camera H51, and a sensor H52. The memory module M is realized by including a drive H7. The memory module M may be configured to further include a part or the whole of a memory H8. The control module P is realized by including a memory H8 and a processor H9. Such hardware constituent elements are connected to be able to communicate with each other through a bus and are supplied with electric power from a power supply H6.

The connection module H12 is a digital input/output port such as a universal serial bus (USB) or the like. In the case of a mobile device, the pointing device H21, the keyboard H22, and the display H23 are configured as a touch panel. The sensor H52 is an acceleration sensor, a gyro sensor, a GPS reception module, a proximity sensor, or the like. The power supply H6 is a power supply unit that supplies electricity required for moving each device. In the case of a mobile device, the power supply H6 is a battery. The drive H7 is an auxiliary memory medium such as a hard disk drive, a solid-state drive, or the like. The drive H7 may be a non-volatile memory such as an EEPROM or a flash memory or a magneto-optical disc drive, or a flexible disk drive. In addition, the drive H7, for example, is not limited to being built into each device and may be a memory device of an external attachment type connected to a connector of the connection module H12. The memory H8 is a main memory medium such as a random access memory (RAM) or the like. In addition, the memory H8 may be a cache memory device. When commands are executed using one or a plurality of processors H9, the memory H8 stores such commands. The processor H9 is a central processing unit (CPU). The processor H9 may be a micro processing unit (MPU) or a graphics processing unit (GPU). By reading a program and various kinds of data from the drive H7 through the memory H8 and performing an arithmetic operation, the processor H9 executes commands stored in one or a plurality of memories H8.

The input/output module I is used in an abnormal waveform extracting unit 301, a pseudo abnormal data output unit 306, an operator terminal 500, and the like. The memory module M realizes a data collecting server 100. The control module P is used for mounting each of units of a data collecting server 100, a learning model generating device 200, a pseudo abnormal data generating device 300, an abnormal sign detecting device 400, and an operator terminal 500. In addition, in this specification and the like, description of the data collecting server 100, the learning model generating device 200, the pseudo abnormal data generating device 300, the abnormal sign detecting device 400, and the operator terminal 500 may be substituted with description of the control module P.

In addition, although each embodiment described above is an example in which the pseudo abnormal data generating device 300 is used in the transmission/distribution network monitoring system 10 monitoring a transmission/distribution network, the pseudo abnormal data generating device 300 may be used in a system other than the transmission/distribution network monitoring system 10. For example, the pseudo abnormal data generating device 300 may be used in a facility system monitoring facilities of a factory, a public facility, and the like, may be used in an observation system observing natural phenomena such as climates and the like, or may be used in a measurement system measuring activities of persons such as humans.

In addition, a group generating unit may be formed from any one of a type classifying unit 302 and a clustering unit 303, and a band generating unit 304 may generate a band for a group of abnormal data classified by the group generating unit.

The following embodiments may be employed.
(1) One embodiment is a pseudo abnormal data generating device including: a group generating unit configured to generate a group including at least a part of a plurality of pieces of abnormal data on the basis of at least feature quantities of the plurality of pieces of abnormal data from the plurality of pieces of abnormal data that is time series data; a band generating unit configured to generate a band representing a range of values including abnormal data included in the group generated by the group generating unit at each time; and a band modulating unit configured to generate pseudo abnormal data on the basis of the band generated by the band generating unit.
(2) Another embodiment is the pseudo abnormal data generating device of (1) described above, in which the band generating unit adjusts positions of pieces of abnormal data included in the group on the basis of feature quantities of the pieces of abnormal data included in the group and generates the band including the pieces of abnormal data of which positions have been adjusted.
(3) Another embodiment is the pseudo abnormal data generating device of (1) or (2) described above, in which the band modulating unit generates the pseudo abnormal data by applying a change to the maximum value or the minimum value of the range of the values represented by the band generated by the band generating unit with the maximum value or the minimum value set as a reference.
(4) Another embodiment is the pseudo abnormal data generating device of (3) described above, in which the band modulating unit performs the change applied to the maximum value or the minimum value at a time of a part of the band.
(5) Another embodiment is a facility monitoring system including: a group generating unit configured to generate a group including at least a part of a plurality of pieces of abnormal data on the basis of at least feature quantities of the plurality of pieces of abnormal data from the plurality of pieces of abnormal data that is time series data; a band generating unit configured to generate a band representing a range of values including abnormal data included in the group generated by the group generating unit at each time; and a band modulating unit configured to generate pseudo abnormal data on the basis of the band generated by the band generating unit.
(6) Another embodiment is the facility monitoring system of (5) described above, in which a learning model generating unit configured to generate a learning model used for detecting an abnormality or a sign of an abnormality from operating data using the pseudo abnormal data and an abnormal sign detecting unit configured to detect an abnormality or a sign of an abnormality from the operating data using the learning model generated by the learning model generating unit are further included.
(7) Another embodiment is the facility monitoring system of (5) or (6) described above, in which a sensor unit configured to measure operating data including the abnormal data is further included.
(8) Another embodiment is the facility monitoring system of (5) or (6) described above, in which a sensor unit configured to measure operating data including the abnormal data and a data collecting unit configured to collect operating data measured by the sensor unit are further included.
(9) Another embodiment is a pseudo abnormal data generating method including: a first step of generating a group including at least a part of a plurality of pieces of abnormal data on the basis of at least feature quantities of the plurality of pieces of abnormal data from the plurality of pieces of abnormal data that is time series data; a second step of generating a band representing a range of values including abnormal data included in the group generated in the first step at each time; and a third step of generating pseudo abnormal data on the basis of the band generated in the second step.
(10) Another embodiment is a program causing a processor to function as: a group generating unit configured to generate a group including at least a part of a plurality of pieces of abnormal data on the basis of at least feature quantities of the plurality of pieces of abnormal data from the plurality of pieces of abnormal data that is time series data; a band generating unit configured to generate a band representing a range of values including abnormal data included in the group generated by the group generating unit at each time; and a band modulating unit configured to generate pseudo abnormal data on the basis of the band generated by the band generating unit.

In addition, by recording a program for realizing the functions of the data collecting server 100, the learning model generating device 200, the pseudo abnormal data generating device 300, the abnormal sign detecting device 400, and the operator terminal 500 illustrated in FIG. 1 on a computer-readable recording medium and causing a computer system to read and execute this program recorded on the recording medium, the data collecting server 100, the learning model generating device 200, the pseudo abnormal data generating device 300, the abnormal sign detecting device 400, and the operator terminal 500 may be realized. In addition, the “computer system” described here is assumed to include an OS and hardware such as peripherals.

In addition, in a case in which a WWW system is used, “computer system” also includes a home page providing environment (or a display environment).

Furthermore, the “computer-readable recording medium” represents a portable medium such as a flexible disk, a magneto-optical disk, a ROM, or CD-ROM or a storage device such as a hard disk built into the computer system. Furthermore, the “computer-readable recording medium” may include a medium dynamically storing the program for a short time such as a communication line of a case in which the program is transmitted through a network such as the Internet or a communication circuit line such as a telephone line and a medium storing the program for a predetermined time such as an internal volatile memory of the computer system that becomes a server or a client in such a case. In addition, the program described above may be a program used for realizing a part of the function described above or a program that can realize the function described above in combination with a program that is already recorded in the computer system.

As above, although the embodiment of the present invention has been described in detail with reference to the drawings, a specific configuration is not limited to that described above, and various design changes and the like can be made in a range not departing from the concept of the present invention.

### REFERENCE SIGNS LIST

10 Transmission/distribution network monitoring system
100 Data collecting server
200 Learning model generating device
300 Pseudo abnormal data generating device
301 Abnormal waveform extracting unit
302 Type classifying unit
303 Clustering unit
304 Band generating unit
305 Band modulating unit
306 Pseudo abnormal data output unit
307 Waveform synthesizing unit
400 Abnormal sign detecting device
500 Operator terminal
600 Network

1. A pseudo abnormal data generating device comprising:
a group generating unit configured to generate a group including at least a part of a plurality of pieces of abnormal data on the basis of at least feature quantities of the plurality of pieces of abnormal data from the plurality of pieces of abnormal data that is time series data;
a band generating unit configured to on a basis of a feature quantity of each of pieces of abnormal data included in the group generated by the group generating unit, adjust positions in a time direction of the pieces of abnormal data included in the group and to generate a band representing a range of values including the adjusted pieces of abnormal data at each time; and
a band modulating unit configured to generate pseudo abnormal data on the basis of the band generated by the band generating unit.

2. The pseudo abnormal data generating device according to claim 1, wherein the band generating unit adjusts positions in a direction of magnitudes of values of the pieces of abnormal data included in the group on the basis of a feature quantity of each of the pieces of abnormal data included in the group and generates the band including the pieces of abnormal data of which positions in the time direction and in the direction of magnitudes of values have been adjusted.

3. The pseudo abnormal data generating device according to claim 1, wherein the band modulating unit generates the pseudo abnormal data by applying a change to a maximum value or a minimum value of the range of the values represented by the band generated by the band generating unit with the maximum value or the minimum value set as a reference.

4. The pseudo abnormal data generating device according to claim 3, wherein the band modulating unit performs the change applied to the maximum value or the minimum value at a time of a part of the band.

5. A facility monitoring system comprising:
a group generating unit configured to generate a group including at least a part of a plurality of pieces of abnormal data on the basis of at least feature quantities of the plurality of pieces of abnormal data from the plurality of pieces of abnormal data that is time series data;
a band generating unit configured to, on a basis of a feature quantity of each of pieces of abnormal data included in the group generated by the group generating unit, adjust positions in a time direction of the pieces of abnormal data included in the group and to generate a band representing a range of values including the adjusted pieces of abnormal data at each time; and
a band modulating unit configured to generate pseudo abnormal data on the basis of the band generated by the band generating unit.

6. The facility monitoring system according to claim 5, further comprising:
a learning model generating unit configured to generate a learning model used for detecting an abnormality or a sign of an abnormality from operating data using the pseudo abnormal data; and
an abnormal sign detecting unit configured to detect an abnormality or a sign of an abnormality from the operating data using the learning model generated by the learning model generating unit.

7. The facility monitoring system according to claim 5, further comprising a sensor unit configured to measure operating data including the abnormal data.

8. The facility monitoring system according to claim 5, further comprising:
a sensor unit configured to measure operating data including the abnormal data; and
a data collecting unit configured to collect operating data measured by the sensor unit.

9. A pseudo abnormal data generating method comprising:
generating a group including at least a part of a plurality of pieces of abnormal data on the basis of at least feature quantities of the plurality of pieces of abnormal data from the plurality of pieces of abnormal data that is time series data;
on a basis of a feature quantity of each of pieces of abnormal data included in the generated group, adjusting positions in a time direction of the pieces of abnormal data included in the group;
generating a band representing a range of values including the adjusted pieces of abnormal data at each time; and
generating pseudo abnormal data on the basis of the generated band

10. A non-transitory computer-readable medium having stored thereon a program causing processor to function as:
a group generating unit configured to generate a group including at least a part of a plurality of pieces of abnormal data on the basis of at least feature quantities of the plurality of pieces of abnormal data from the plurality of pieces of abnormal data that is time series data;
a band generating unit configured to, on a basis of a feature quantity of each of pieces of abnormal data included in the group generated by the group generating unit, adjust positions in a time direction of the pieces of abnormal data included in the group and to generate a band representing a range of values including the adjusted pieces of abnormal data included in the group at each time; and
a band modulating unit configured to generate pseudo abnormal data on the basis of the band generated by the band generating unit.

11. The pseudo abnormal data generating device according to claim 2, wherein the band modulating unit generates the pseudo abnormal data by applying a change to a maximum value or a minimum value of the range of the values represented by the band generated by the band generating unit with the maximum value or the minimum value set as a reference.

12. The pseudo abnormal data generating device according to claim 11, wherein the band modulating unit performs the change applied to the maximum value or the minimum value at a time of a part of the band.

13. The facility monitoring system according to claim 6, further comprising a sensor unit configured to measure operating data including the abnormal data.

14. The facility monitoring system according to claim 6, further comprising:
a sensor unit configured to measure operating data including the abnormal data; and
a data collecting unit configured to collect operating data measured by the sensor unit.
